(19) United States
(12) Patent Application Publication
(10) Pub. No.: US 2011/0124551 A1
(43) Pub. Date: May 26, 2011

(54) FORMULATIONS OF DAPTOMYCIN

(75) Inventors: Nagesh R. Palepu, Southampton, PA (US); Bulusu Bhanu Teja, Hyderabad (IN)

(73) Assignee: EAGLE PHARMACEUTICALS, INC., Woodcliff Lake, NJ (US)

(21) Appl. No.: 12/885,009

(22) Filed: Sep. 17, 2010

Related U.S. Application Data
(60) Provisional application No. 61/263,695, filed on Nov. 23, 2009.
(60) Provisional application No. 61/371,802, filed on Aug. 9, 2010.

(51) Int. Cl. A61K 38/12; A61P 7/00; A61P 17/00; A61P 31/04

(52) U.S. Cl. 514/2.9

(57) ABSTRACT

Long term storage stable daptomycin-containing compositions are disclosed. The compositions include a pharmacologically acceptable fluid including daptomycin or a pharmaceutically acceptable salt thereof at a concentration of less than or equal to about 25 mg/mL, and a calcium source. The formulations are surprisingly free of degradation products such as the hydrolysis product of daptomycin, the β-isomer of daptomycin and anhydro daptomycin after storage periods of at least about 18 months under refrigerated conditions.

### CROSS-REFERENCE TO RELATED APPLICATIONS

This application claims priority under 35 U.S.C. §119(e) to U.S. Provisional Patent Application No. 61/263,695, filed Nov. 23, 2009, entitled “FORMULATIONS OF DAPTOMYCIN”, and U.S. Provisional Patent Application No. 61/371,802, filed Aug. 9, 2010, entitled “FORMULATIONS OF DAPTOMYCIN”, the disclosures of each of which are incorporated by reference herein in their entirety.

### BACKGROUND OF THE INVENTION

Daptomycin is a lipopeptide antibiotic represented by the following structural formula (I)

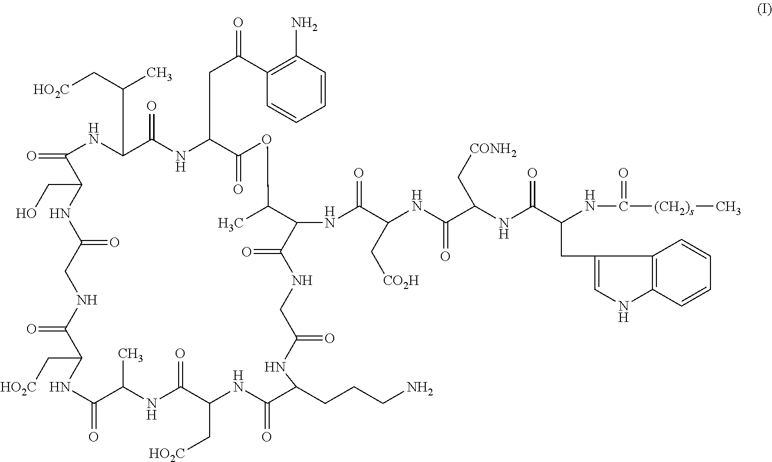

and is described, for example, in U.S. Pat. No. 4,537,717, the contents of which are incorporated herein by reference.

Daptomycin is used in the treatment of Methicillin-resistant Staphylococcus aureus (MRSA) and Methicillin-susceptible Staphylococcus aureus (MSSA), Streptococcus pyogenes, Streptococcus agalactiae, Streptococcus dysgalactiae subspecies equisimilis, and Enterococcus faecalis (vancomycin-susceptible isolates only) in complicated skin infections and bloodstream infections (bacteremia), including right-sided infective endocarditis. Daptomycin is commercially available as Cubicin™ for intravenous administration.

Daptomycin exhibits premature degradation upon reconstitution of the lyophilized product. The reconstituted daptomycin exhibits increased degradation after reconstitution and is, therefore, not suitable for long-term storage in liquid form. Some of the main degradants of daptomycin are the hydrolysis product of daptomycin, the β-isomer of daptomycin and anhydro daptomycin. The hydrolysis product (ring opening compound) appears as the main impurity at a Relative Retention Time (RRT) of about 0.66, the β-isomer of daptomycin appears as the main impurity at an RRT of about 0.97 and anhydro daptomycin appears as the main impurity at an RRT of about 1.1. There is a need for daptomycin formulations with increased stability.

### SUMMARY OF THE INVENTION

The invention is generally directed to daptomycin-containing compositions that are long term storage stable, i.e. for a period of at least about 18 months or longer. In several aspects of the invention, the compositions include a source of calcium selected from among calcium chloride and calcium lactate and the concentration of daptomycin will be less than or equal to about 25 mg/mL. In other aspects of the invention, the compositions will have a pH of from about 6 to about 7. In other aspects of the invention, the compositions will include an alkaline hydroxide selected from among calcium hydroxide, magnesium hydroxide and aluminum hydroxide. Still further aspects of the invention include methods of treatment, methods of preparing the compositions and kits.

In other aspects of the invention, the compositions will include an amino acid in an amount sufficient to maintain the pH of the composition at about 6 to about 7. In other aspects of the invention, the compositions will include trehalose.

One of the advantages of the inventive liquid compositions is that they are substantially free of impurities after at least 18 months. Substantially free of impurities refers to daptomycin-containing compositions in which total impurities are less than about 10%, including less than about 5% of the hydrolysis product of daptomycin, less than about 5% of the β-isomer of daptomycin and less than 5% of anhydro-daptomycin, area-under-the-curve (“AUC”) as determined by high performance liquid chromatography (“HPLC”) at a wavelength of 223 nm, after a period of at least about 18 months at a temperature of from about 5° C. to about 25° C. The formulations are ready for use or further dilution; storage as a lyophilized powder is no longer a necessity for commercial use of the drug.

### DETAILED DESCRIPTION OF THE INVENTION

Unless defined otherwise, all technical and scientific terms used herein have the same meaning as is commonly understood by one of ordinary skill in the art to which this invention belongs. In the event that there are a plurality of definitions for a term herein, those in this section prevail unless stated otherwise.

As used herein, RRT is calculated by dividing the retention time of the peak of interest by the retention time of the main peak. Any peak with an RRT<1 elutes before the main peak, and any peak with an RRT>1 elutes after the main peak.

As used herein, substantially free of impurities refers to daptomycin-containing compositions in which the total impurities are less than about 10%, calculated as being based upon the original amount daptomycin (or salt thereof) being present in the composition or formulation. Preferably, the total amount of impurities, i.e. >10% includes less than about 5%, i.e. no more than about ⅓ thereof, of the hydrolysis product of daptomycin, less than about 5%, i.e. no more than about ⅓ thereof, of the β-isomer of daptomycin and less than 5%, i.e. no more than about ⅓ thereof, of anhydro-daptomycin. The amounts of impurities are calculated as area-under-the-curve (“AUC”) as determined by high performance liquid chromatography (“HPLC”) at a wavelength of 223 nm, after storage periods of at least about 18 months at a temperature of from about 5° C. to about 25° C. In preferred aspects the amount of time the compositions demonstrate long term storage stability is at least about 2 years.

In accordance with one aspect of the invention, there are provided long term storage stable daptomycin-containing compositions, including:
a) daptomycin or a pharmaceutically acceptable salt thereof at a concentration of less than or equal to about 25 mg/mL; and
b) a source of calcium selected from among calcium chloride and calcium lactate. The compositions have a pH of from about 6 to about 7, and total impurities are less than about 10% area-under-the-curve (“AUC”) as determined by high performance liquid chromatography (“HPLC”) at a wavelength of 223 nm, after at least about 18 months storage at a temperature of from about 5° C. to about 25° C.

In most of the embodiments described herein, it will be understood that when the daptomycin-containing compositions described herein are referred to as having total impurities of less than about 10%, the compositions will further include less than about 5% of the hydrolysis product of daptomycin, less than about 5% of the β-isomer of daptomycin and less than 5% of anhydro-daptomycin (as calculated with reference to the original starting amount of daptomycin) after the same period of long term storage, i.e. about 18 months or longer under the conditions mentioned herein.

In some aspects of the invention, the source of calcium is calcium chloride. In some embodiments, the amount of calcium chloride is greater than 1 mg/mL. In other aspects of the invention, the concentration of calcium chloride is from about 1.5 mg/mL to about 17 mg/mL. In one embodiment, the concentration of calcium chloride is from about 4 mg/mL to about 16.2 mg/mL. In another embodiment, the concentration of calcium chloride is from about 8 mg/mL to about 12 mg/mL. Some preferred concentrations of calcium chloride include, for example, 8 mg/mL, 12 mg/mL or 16.2 mg/mL.

In other aspects of the invention, the source of calcium is calcium lactate. In some aspects, the calcium lactate concentration is about 0.05M to about 0.3M. Preferably, the concentration of calcium lactate is about 0.1M to about 0.25M. More preferably, the concentration of calcium lactate is about 0.1M.

The compositions of the present invention can be kept at a pH of from about 6.25 to about 6.75. Preferably, the composition is maintained at a pH of from about 6.5 to about 6.75. In one embodiment, the pH is about 6.5. In another embodiment, the pH is about 6.75.

In other aspects of the invention, the long term storage stable daptomycin-containing compositions include a pH adjusting agent which is present in an amount sufficient to adjust the pH of the compositions to the ranges set forth above, i.e. from about 6.25 to about 6.75, or to specific points in between such as about 6.5 or about 6.75. One preferred pH adjusting agent is sodium hydroxide. Another preferred pH adjusting agent is calcium hydroxide. Alternative pH adjusters are those commonly used in the art, including HC1 and TRIS.

Without meaning to be bound by any theory or hypothesis, it is been surprisingly found that daptomycin is predominantly ionized at pHs of from about 6.5 to about 7.0. As a result the molecule is considerably more stable and thus self association and degradation thereof is unexpectedly and substantially reduced for extended periods of time.

In some aspects of the invention, the inventive compositions are maintained during storage and/or prior to use at a temperature of from about 5° C. to about 15° C. In another embodiment, the compositions are maintained at a temperature of from about 5° C. to about 10° C. More preferably, the compositions are maintained at a temperature of about 5° C., i.e. at about refrigerated temperatures and conditions.

The amount of daptomycin included in the compositions of the present invention is generally in concentrations of from about 1 mg/mL to about 25 mg/mL. In another embodiment of the invention, the daptomycin concentration is from about 5 mg/mL to about 20 mg/mL. In yet another embodiment, the daptomycin concentration is from about 7.5 mg/mL to about 15 mg/mL. In a further embodiment of the invention, the daptomycin concentration is from about 10 mg/mL to about 15 mg/mL. Preferably, the daptomycin concentration is about 10 mg/mL.

The compositions of the present invention can also include an alkaline hydroxide selected from among calcium hydroxide, magnesium hydroxide and aluminum hydroxide. Preferably, the alkaline hydroxide is calcium hydroxide. In some aspects, the alkaline hydroxide concentration is from about 0.5 mg/ml to about 1 mg/ml. Preferably, the alkaline hydroxide concentration is about 0.68 mg/ml.

Without meaning to be bound by any theory or hypothesis, it is thought that the presence of a counter ion, i.e., calcium source, alkaline hydroxide, amino acid, stabilizes daptomycin and slows down degradation.

A further embodiment of the invention includes long term storage stable daptomycin-containing compositions which include:
a) daptomycin or a pharmaceutically acceptable salt thereof at a concentration of less than or equal to about 25 mg/mL; and
b) calcium chloride at a concentration of greater than 1 mg/mL. These compositions have a pH of from about 6 to about 7; and have the same stability profiles as already described, i.e. having total impurities of less than about 10%, less than about 5% AUC of the hydrolysis product of daptomycin, less than about 5% AUC of the β-isomer of daptomycin and less than about 5% AUC of anhydro-daptomycin, area-under-the-curve (“AUC”) as determined by high performance liquid chromatography (“HPLC”) at a wavelength of 223 nm, after at least about 18 months storage at a temperature of from about 5° C. to about 25° C.

Other compositions in accordance with the present invention include:
a) daptomycin or a pharmaceutically acceptable salt thereof at a concentration of less than or equal to about 25 mg/mL; and
b) 0.1 M calcium lactate. Unlike the pH ranges recited immediately above, the compositions in this embodiment have a pH of about 6.5. The stability profile is the same as previously mentioned, i.e. having less than about 10% total impurities, etc.

A further embodiment of the invention includes daptomycin-containing compositions having similar long term stability profiles, but includes:
a) daptomycin or a pharmaceutically acceptable salt thereof at a concentration of less than or equal to about 25 mg/mL;
b) calcium chloride at a concentration of about 16.2 mg/mL; and
c) calcium hydroxide. The pH of these compositions is from about 6 to about 7 and the impurity profile is the same as that mentioned above.

Another embodiment of the invention includes methods of treating a daptomycin sensitive disease in mammals. The methods include administering, to a mammal in need thereof, an effective amount of a daptomycin-containing composition described herein. Since the active ingredient portion of the inventive compositions is an FDA-approved drug, those of ordinary skill will recognize that the doses of daptomycin employed in this aspect of the invention will be the similar to those employed in any treatment regimens designed for daptomycin as marketed under the trade name Cubicin™. The patient package insert containing dosing information is incorporated herein by reference. The methods of treatment also include administering the inventive formulations for any purpose or physical condition for which daptomycin has been indicated as being useful.

Another embodiment of the invention includes methods of preparing daptomycin-containing compositions described herein. The methods include reconstituting lyophilized daptomycin to a concentration of less than or equal to about 25 mg/mL in a pharmacologically acceptable fluid including a source of calcium selected from among calcium chloride and calcium lactate, and adjusting the pH of the composition to about 6.0 to about 7.0. The steps are carried out under pharmaceutically acceptable conditions for sterility and manufacturing. The reconstitution of the daptomycin can also be carried out with the buffer and a sufficient amount of an aqueous solution.

In a further aspect of the invention, there are provided methods of controlling or preventing the formation of impurities in daptomycin-containing compositions during long term storage. The methods include combining an amount of daptomycin or a pharmaceutically acceptable salt thereof with a sufficient amount of a pharmacologically acceptable fluid including a source of calcium selected from among calcium chloride and calcium lactate so that a formulation or composition is formed wherein the amount daptomycin or pharmaceutically acceptable salt thereof included therein is at a concentration of less than or equal to about 25 mg/ml and the pH of the resultant formulation is from about 6.0 to about 7.0. Further optional steps in accordance therewith include transferring one or more pharmaceutically acceptable doses of the formulations into a suitable sealable container and storing the sealed container at a temperature of from about 5° C. to about 25° C. As a result of carrying out these steps, it is possible to control or substantially prevent the formation of impurities which otherwise occur with daptomycin-containing compositions during long term storage so that the artisan is provided with daptomycin-containing formulations having less than about 10% total impurities area-under-the-curve (“AUC”) as determined by high performance liquid chromatography (“HPLC”) at a wavelength of 223 nm, after at least about 18 months of storage at a temperature of from about 5° C. to about 25° C. The method described herein provides compositions or formulations in which the less than about 10% total impurities is comprised of less than about 5% AUC of the hydrolysis product of daptomycin, less than about 5% AUC of the β-isomer of daptomycin and less than about 5% AUC of anhydro-daptomycin, based on the initial amount daptomycin included in the composition.

The compositions of the present invention can be packaged in any suitable sterile vial or container fit for the sterile storage of a pharmaceutical such as daptomycin. Suitable containers can be glass vials, polypropylene or polyethylene vials or other special purpose containers and be of a size sufficient to hold one or more doses of daptomycin.

A further aspect of the invention includes a kit containing the daptomycin-containing compositions described herein. As will be appreciated by those of ordinary skill, the kit will contain at least one pharmaceutically acceptable vial or container containing one or more doses of the daptomycin-containing compositions as well as other pharmaceutically necessary materials for storing and/or administering the drug, including instructions for storage and use, infusion bag or container with normal saline or D₅W, additional diluents, if desired, etc.

In accordance with another aspect of the invention, there are provided long term storage stable daptomycin-containing compositions including:
a) daptomycin or a pharmaceutically acceptable salt thereof at a concentration of less than or equal to about 25 mg/mL; and
b) an alkaline hydroxide selected from among calcium hydroxide, magnesium hydroxide and aluminum hydroxide, in an amount sufficient to maintain the composition at a pH of from about 6 to about 7. Total impurities of the daptomycin-containing compositions are less than about 10% area-under-the-curve (“AUC”) as determined by high performance liquid chromatography (“HPLC”) at a wavelength of 223 nm, after storage for at least about 18 months at a temperature of from about 5° C. to about 25° C. As with the embodiments described above, it will be understood that when the daptomycin-containing compositions described herein are referred to as having total impurities of less than about 10%, the compositions will further include less than about 5% AUC of the hydrolysis product of daptomycin, less than about 5% AUC of the β-isomer of daptomycin and less than about 5% AUC of anhydro-daptomycin (as calculated with reference to the original starting amount of daptomycin) after the same period of long term storage, i.e. about 18 months or longer under the conditions mentioned herein.

In some aspects of the invention, the amount of alkaline hydroxide is sufficient to maintain a pH of from about 6.5 to about 6.75. Preferably, the amount of alkaline hydroxide is sufficient to maintain a pH of about 6.75.

In accordance with yet another aspect of the invention, there are provided long term storage stable daptomycin-containing compositions including:
a) daptomycin or a pharmaceutically acceptable salt thereof at a concentration of less than or equal to about 25 mg/mL; and
b) an amino acid in an amount sufficient to maintain the pH of the composition at about 6 to about 7. These compositions have the same stability profiles as already described, i.e., having less than about 10% area-under-the-curve (“AUC”) as determined by high performance liquid chromatography (“HPLC”) at a wavelength of 223 nm, after at least about 18 months storage at a temperature of from about 5° C. to about 25° C.

In some aspects of the invention, the amino acid is selected from among arginine, glycine, alanine, valine, methionine and histidine. More preferably, the amino acid is arginine.

In other aspects of the present invention, the amino acid is present in an amount sufficient to maintain the pH of the compositions in the ranges set forth above, i.e. from about 6.5 to about 6.75. Preferably, the amount of amino acid is sufficient to maintain a pH of about 6.75.

In some aspects of the invention, the inventive compositions are maintained during storage and/or prior to use at a temperature of from about 5° C. to about 15° C. Preferably, the compositions are maintained at a temperature of from about 5° C. to about 10° C. More preferably, the compositions are maintained at a temperature of about 5° C., i.e. about refrigerated conditions.

The amount of daptomycin included in the compositions of the present invention is generally concentrations of from about 1 mg/mL to about 25 mg/mL. In another embodiment of the invention, the daptomycin concentration is from about 10 mg/mL to about 25 mg/mL. Alternatively, it can be from about 5 mg/mL to about 20 mg/mL. In yet another embodiment, the daptomycin concentration is about 7.5 mg/mL to about 15 mg/mL. Preferably, the daptomycin concentration is about 10 mg/mL.

Some preferred embodiments of the invention include daptomycin-containing compositions in which the total amount of impurities is less than about 8% and more preferably less than about 6% area-under-the-curve (“AUC”) as determined by high performance liquid chromatography (“HPLC”) at a wavelength of 223 nm after a storage period of about 18 months at a temperature of from about 5° C. to about 25° C.

The compositions of the present invention can be self preserved to maintain stability and sterility. Alternatively, the compositions can include one or more art recognized stabilizers and/or preservatives in amounts generally recognized as being effective for such purposes. The stabilizer can be selected from among trehalose, sucrose and hetastarch. Preferably, the stabilizer is trehalose. In some aspects of the invention, the stabilizer content is less than 4 g/mL. Preferably, the stabilizer content is from about 10 mg/mL to about 200 mg/mL. More preferably, the stabilizer content is from about 10 mg/mL to about 50 mg/mL.

In other aspects of the invention, the compositions include a tonicifying agent, in amounts which preferably render the composition isotonic or substantially isotonic. Some preferred tonicifying agents include glycerin, sodium chloride, polyethylene glycol (PEG) 400, propylene glycol or injectable grade polyvinylpyrrolidone (PVP). More preferably, the tonicifying agent is polyethylene glycol (PEG) 400. In some aspects of the invention, the tonicifying agent content in the compositions is about 2.5% (v/v) to about 5.0% (v/v). Preferably, the tonicifying agent content is about 5% (v/v).

A further embodiment of the invention includes long term storage stable daptomycin-containing compositions which include:
a) daptomycin or a pharmaceutically acceptable salt thereof at a concentration of less than or equal to about 25 mg/mL;
b) an alkaline hydroxide selected from among calcium hydroxide, magnesium hydroxide and aluminum hydroxide, in an amount sufficient to maintain the composition at a pH of from about 6 to about 7; and
c) an amino acid in an amount sufficient to maintain the pH of the composition at about 6 to about 7. The stability profile is the same as previously mentioned, i.e. having less than about 10% total impurities, etc.

A further embodiment of the invention provides a long term storage stable daptomycin-containing compositions, including a pharmacologically acceptable fluid which includes:
a) daptomycin or a pharmaceutically acceptable salt thereof at a concentration of about 10 mg/mL;
b) calcium hydroxide in an amount sufficient to maintain the composition at a pH of about 6.75;
c) arginine in an amount sufficient to maintain the composition at a pH of about 6.75; and
d) 5% trehalose. The impurity profile is the same as that mentioned above.

Another embodiment of the invention includes methods of treating daptomycin sensitive disease in mammals. The methods include administering, to a mammal in need thereof, an effective amount of daptomycin-containing compositions described herein. As described above, the methods of treatment also include administering the inventive formulations for any purpose or physical condition for which daptomycin has been indicated as being useful.

Another aspect of the invention provides kits including lyophilized daptomycin, and pharmacologically suitable fluids with calcium hydroxide in an amount sufficient to maintain the composition at a pH of about 6.75, arginine in an amount sufficient to maintain the composition at a pH of about 6.75, and about 5% trehalose.

Lyophilizates of daptomycin and about 5% trehalose are provided in another embodiment of the invention. In a further embodiment, kits include the lyophilizate of daptomycin and about 5% trehalose and a pharmacologically suitable fluid with calcium hydroxide in an amount sufficient to maintain the composition at a pH of about 6.75 and arginine in an amount sufficient to maintain the composition at a pH of about 6.75.

In a further embodiment of the invention, there are provided methods of preparing daptomycin-containing compositions including reconstituting lyophilized daptomycin, to a concentration of less than or equal to about 25 mg/mL in a pharmacologically suitable fluid including alkaline hydroxides selected from among calcium hydroxide, magnesium hydroxide and aluminum hydroxide, in an amount sufficient to maintain the composition at a pH of from about 6 to about 7 and/or an amino acid in an amount sufficient to maintain the pH of the composition at about 6 to about 7. In some aspects the lyophilizate includes daptomycin and 5% trehalose. In other aspects of the invention, the pharmacologically suitable fluid also contains trehalose. The steps are carried out under pharmaceutically acceptable conditions for sterility and manufacturing. The reconstitution of the daptomycin can also be carried out with the buffer and a sufficient amount of an aqueous solution.

In a further aspect of the invention, there are provided methods of controlling or preventing the formation of impurities in daptomycin-containing compositions during long term storage. The methods include combining an amount of daptomycin or a pharmaceutically acceptable salt thereof with a sufficient amount of a pharmacologically suitable fluid including alkaline hydroxides selected from among calcium hydroxide, magnesium hydroxide and aluminum hydroxide, in an amount sufficient to maintain the composition at a pH of from about 6 to about 7 and/or an amino acid in an amount sufficient to maintain the pH of the composition at about 6 to about 7 so that a formulation or composition is formed wherein the amount daptomycin or pharmaceutically acceptable salt thereof included therein is at a concentration of less than or equal to about 25 mg/ml. Further optional steps in accordance therewith include transferring one or more pharmaceutically acceptable doses of the formulations into a suitable sealable container and storing the sealed container at a temperature of from about 5° C. to about 25° C. As a result of carrying out these steps, it is possible to control or substantially prevent the formation of impurities which otherwise occur with daptomycin-containing compositions during long term storage so that the artisan is provided with daptomycin-containing formulations having less than about 10% total impurities area-under-the-curve (“AUC”) as determined by high performance liquid chromatography (“HPLC”) at a wavelength of 223 nm, after at least about 18 months of storage at a temperature of from about 5° C. to about 25° C. The method described herein provides compositions or formulations in which the less than about 10% total impurities is comprised of less than about 5% AUC of the hydrolysis product of daptomycin, less than about 5% AUC of the β-isomer of daptomycin and less than about 5% AUC of anhydro-daptomycin, based on the initial amount daptomycin included in the composition.

The compositions of the present invention can be packaged in any suitable sterile vial or container fit for the sterile storage of a pharmaceutical such as daptomycin. Suitable containers can be glass vials, polypropylene or polyethylene vials or other special purpose containers and be of a size sufficient to hold one or more doses of daptomycin.

A further aspect of the invention includes a kit containing the daptomycin-containing compositions described herein. As will be appreciated by those of ordinary skill, the kit will contain at least one pharmaceutically acceptable vial or container containing one or more doses of the daptomycin-containing compositions as well as other pharmaceutically necessary materials for storing and/or administering the drug, including instructions for storage and use, infusion bag or container with normal saline or D₅W, additional diluents, if desired, etc.

### EXAMPLES

The following examples serve to provide further appreciation of the invention but are not meant in any way to restrict the effective scope of the invention.

### Example 1

Daptomycin-containing compositions were prepared by dissolving daptomycin in distilled water to obtain a daptomycin concentration of 10 mg/mL. The “control” sample was prepared by adding a sufficient amount of NaOH to obtain a pH of 6.75. The “Ca(OH)₂” sample was prepared by adding a sufficient amount of a 0.5% Ca(OH)₂ dispersion to obtain a pH of 6.75. The “arginine” sample was prepared by adding a sufficient amount of arginine to obtain a pH of 6.75. The “5% Trehalose” sample was prepared by adding Trehalose to a sample prepared in the same manner as the control to obtain a 5% (v/v) solution. The “5% PEG 400” sample was prepared by adding PEG 400 to a sample prepared in the same manner as the control to obtain a 5% (v/v) solution. The samples were stored at 5° C.

The samples were tested for impurities after initial preparation, and again as indicated in Table 1. The samples were tested via HPLC at a wavelength of 223 nm, and the amount of daptomycin in the initial sample and the area % for ring opening products, β-daptomycin and total degradants were measured obtain the area-under-the-curve (“AUC”) after storage. The area % of total degradants was used to determine the change in area %. The test data is reported in Table 1 below.

TABLE 1
Stability of Daptomycin (10 mg/mL), pH 6.75 With and
Without Ca(OH)₂ at 5° C.

| | | | Area % of | | | |
|---|---|---|---|---|---|---|
| | | | Ring | | | |
| | | | Opening | Area | Total | Change |
| | | Time | Products | % of | Imp. | in |
| Formulation | Temp. | Period | (ROP) | β-Isomer | % | Area % |
| Control | Initial | | 0.39 | 0.94 | 3.80 | |
| | 5° C. | 1 M | 0.69 | 1.43 | 4.59 | 0.79 |
| | | 2 M | 0.61 | 1.93 | 4.74 | 0.94 |
| | | 3 M | 0.84 | 2.46 | 6.27 | 2.47 |
| | | 4 M | 1.74 | 3.47 | 7.49 | 3.69 |
| pH adj. | Initial | | 0.49 | 1.05 | 3.91 | |
| Ca(OH)₂ | 5° C. | 1 M | 0.71 | 1.78 | 4.96 | 1.05 |
| | | 2 M | 0.56 | 1.85 | 4.85 | 0.94 |
| | | 3 M | 0.99 | 2.09 | 5.12 | 1.21 |
| | | 4 M | 1.59 | 2.45 | 5.37 | 1.46 |
| pH adj. | Initial | | 0.33 | 0.80 | 3.33 | |
| arginine | 5° C. | 1 M | 0.85 | 1.75 | 4.32 | 0.99 |
| | | 2 M | 0.76 | 1.98 | 4.82 | 1.49 |
| | | 3 M | 1.12 | 2.97 | 6.27 | 2.94 |
| | | 4 M | 1.05 | 2.72 | 6.2 | 2.87 |
| 5% | Initial | | 0.33 | 0.77 | 3.27 | |
| Trehalose | 5° C. | 1 M | 0.82 | 1.68 | 3.76 | 0.49 |
| | | 2 M | 0.90 | 2.25 | 5.33 | 2.06 |
| | | 3 M | 1.13 | 2.50 | 5.26 | 1.99 |
| | | 4 M | 1.13 | 2.49 | 5.94 | 2.67 |
| 5% PEG | Initial | | 0.32 | 0.85 | 3.54 | |
| 400 | 5° C. | 1 M | 0.74 | 1.55 | 4.69 | 1.15 |
| | | 2 M | 0.65 | 2.02 | 4.88 | 1.34 |
| | | 3 M | 0.90 | 2.49 | 6.42 | 2.88 |
| | | 4 M | 1.71 | 3.59 | 7.51 | 3.97 |

As shown in Table 1, the daptomycin formulations are stable in solutions containing calcium hydroxide. Table 1 shows that daptomycin, when reconstituted at a concentration of about 10 mg/mL, in a solution containing an alkaline hydroxide, such as calcium hydroxide, in an amount sufficient to maintain the composition at a pH of about 6.75, and stored at 5° C., had substantially no increase in total degradants. Table 1 shows that the samples formulated with an alkaline hydroxide, such as calcium hydroxide, in an amount sufficient to maintain the composition at a pH of about 6.75, had 1.46% total impurities after 4 months analysis at 5° C. The data presented in Table 1 translates to daptomycin-containing compositions including an alkaline hydroxide having a shelf life of at least about 18 months under refrigerated conditions with levels of impurities within the levels required herein.

The daptomycin formulations are also stable in solutions containing arginine. The 5% trehalose solution also provided sufficient long-term stabilizing effects. Table 1 shows that the formulations made with arginine and trehalose had less than a 3% increase in total degradants at the end of four months analysis at 5° C. The data presented in Table 1 translates to daptomycin-containing compositions including an amino acid, or trehalose having a shelf life of at least about 18 months under refrigerated conditions with levels of impurities within the levels required herein.

As shown in Table 1, the control sample, which was pH adjusted with NaOH, and the sample containing PEG 400 did not provide such stabilizing effects. These samples exhibited an increase of more than 3.5% total degradant peak area compared to initial after 4 months analysis at 5° C. Daptomycin-containing compositions with such high levels of degradation would not be suitable for long-term storage.

### Example 2

Daptomycin-containing compositions were prepared by dissolving daptomycin (“DPT”) in distilled water to obtain a daptomycin concentration of 10 mg/mL and by adding a sufficient amount of a 0.5% Ca(OH)₂ dispersion to obtain a pH of 6.75 or 6.5 as indicated in Table 2. The samples were stored at the temperatures indicated in Table 2 below.

Samples were tested for impurities after initial preparation, and again as indicated in Table 2. The samples were tested via HPLC, at a wavelength of 223 nm, and the amount of daptomycin in the initial sample and the relative retention times (“RRT”) for each of the hydrolysis product of daptomycin (0.66), the β-isomer of daptomycin (0.97) and anhydro-daptomycin (1.1) were added to obtain the total impurities area-under-the-curve (“AUC”) after storage. The test data is reported in Table 2 below.

TABLE 2
Stability of Daptomycin (10 mg/mL) in presence of Ca(OH)₂

| | | Time | | Conc. | % of | | | | | Total | |
|---|---|---|---|---|---|---|---|---|---|---|---|
| Formulation | Temp. | Period | | (mg/mL) | Initial | ΣROP | β-Isomer | AH DPT | Σunknown | Imp. % | pH |
| DPT - | Initial | | | 10.0 | 100.0 | 0.52 | 0.83 | 1.23 | 0.95 | 3.53 | 6.67 |
| 10 mg/mL | 10° C. | 15 | d | 9.94 | 99.4 | 0.48 | 1.18 | 0.76 | 0.99 | 3.41 | 6.53 |
| Water qs to | | 1 | M | 9.82 | 98.2 | 0.45 | 1.48 | 0.71 | 0.82 | 3.46 | 6.07 |
| 1 mL | | 1.5 | M | 9.81 | 98.1 | 0.58 | 1.76 | 0.72 | 0.87 | 3.93 | 7.00 |
| pH - 6.75 | | 2 | M | 9.82 | 98.2 | 0.96 | 2.48 | 0.86 | 0.97 | 5.27 | 6.02 |
| with | | 2.5 | M | 9.83 | 98.3 | 0.80 | 2.63 | 0.92 | 1.22 | 5.57 | 6.04 |
| Ca(OH)₂ | | 3 | M | 9.53 | 95.3 | 1.03 | 3.48 | 1.06 | 1.25 | 6.82 | 6.07 |
| | 5° C. | 1 | M | 10.0 | 100.0 | 0.33 | 1.08 | 0.87 | 0.97 | 3.25 | 5.93 |
| | | 2 | M | 9.86 | 98.6 | 0.62 | 1.57 | 1.01 | 0.73 | 3.93 | 6.09 |
| | | 3 | M | 9.60 | 96.0 | 0.68 | 1.91 | 0.74 | 1.15 | 4.48 | 6.13 |
| DPT - | Initial | | | 9.91 | 100.0 | 0.57 | 0.81 | 1.17 | 0.49 | 3.04 | 6.54 |
| 10 mg/mL | 10° C. | 1.5 | M | 9.54 | 96.3 | 0.41 | 1.90 | 1.16 | 1.33 | 5.01 | 6.55 |
| Water qs to | | 2 | M | 8.87 | 89.5 | 0.80 | 2.67 | 1.48 | 1.29 | 6.24 | 6.59 |
| 1 mL | | 2.5 | M | 8.46 | 85.4 | 0.83 | 3.16 | 1.37 | 1.31 | 6.67 | 6.52 |
| pH - 6.5 | | 3 | M | 8.57 | 86.5 | 0.74 | 3.57 | 1.21 | 1.77 | 7.29 | 6.56 |
| with | 5° C. | 1 | M | 9.80 | 98.9 | 0.42 | 1.16 | 1.17 | 1.32 | 4.07 | 6.46 |
| Ca(OH)₂ | | 2 | M | 9.74 | 98.3 | 0.59 | 1.64 | 1.18 | 1.09 | 4.50 | 6.54 |
| | | 3 | M | 9.68 | 97.7 | 0.49 | 1.99 | 1.07 | 1.53 | 5.08 | 6.51 |

As shown in Table 2, the Ca(OH)₂ stabilized the daptomycin-containing solutions between pH 6.5 and 6.75. The area % of the total impurities increased about 0.95% at a pH of 6.75 over three months analysis at 5° C. Such an increase projects a shelf-life of about 24 months under refrigerated conditions within the levels required herein.

The area % of the total impurities increased about 2.04% at a pH of 6.5 over three months analysis at 5° C., which projects a shelf-life of about 18 months under refrigerated conditions with levels of impurities within the levels required herein.

### Example 3

Daptomycin-containing compositions were prepared by dissolving daptomycin (“DPT”) in distilled water to obtain a daptomycin concentration of 10 mg/mL and by adding a filtrate of a 0.7 mg/ml Mg(OH)₂ solution. 0.1M Ca(OH)₂ was added to the solutions to obtain a pH as indicated in Table 3. The samples were stored at the temperatures indicated in Table 3 below.

Samples were tested for impurities after initial preparation, and at times indicated in Table 3. The samples were tested via HPLC, at a wavelength of 223 nm, and the amount of daptomycin in the initial sample and the relative retention times (“RRT”) for each of the hydrolysis product of daptomycin (0.66), the β-isomer of daptomycin (0.97) and anhydro-daptomycin (1.1) were added to obtain the total impurities area-under-the-curve (“AUC”) after storage. The test data is reported in Table 3 below.

TABLE 3
Stability of Daptomycin (10 mg/mL) in presence of Mg(OH)₂ and Ca(OH)₂

| | | Time | | Conc. | % of | RRTs of Degradants | | | | % of | pH Value |
|---|---|---|---|---|---|---|---|---|---|---|---|
| Formulation | Temp. | Period | | (mg/mL) | Initial | ΣROP | β | AH DPT | Σunknown | Total | Sample |
| DPT - | Initial | | | 10.8 | 100 | 0.12 | 0.69 | 1.08 | 0.88 | 2.77 | 6.56 |
| 10 mg/mL | 10° C. | 0.5 | M | 10.8 | 100 | 0.46 | 1.27 | 1.08 | 0.73 | 3.54 | 6.57 |
| Magnesium | | 1 | M | 10.4 | 96.3 | 0.30 | 1.57 | 1.27 | 1.51 | 4.65 | 6.58 |
| hydroxide | | 1.5 | M | 10.4 | 96.3 | 0.45 | 1.92 | 1.04 | 1.16 | 4.57 | 6.60 |
| Calcium | | 2 | M | 10.2 | 94.4 | 0.58 | 2.52 | 1.10 | 1.33 | 5.53 | 6.57 |
| hydroxide | | 2.5 | M | 10.1 | 93.5 | 0.59 | 2.92 | 1.10 | 0.70 | 5.31 | 6.52 |
| pH - 6.50 | | 3 | M | 10.2 | 94.4 | 1.07 | 3.60 | 1.57 | 1.21 | 7.45 | 6.56 |
| | 5° C. | 1 | M | 10.8 | 100 | 0.21 | 1.04 | 0.99 | 0.84 | 3.08 | 6.55 |
| | | 2 | M | 10.5 | 97.2 | 0.26 | 1.52 | 0.98 | 1.16 | 3.92 | 6.58 |
| | | 3 | M | 10.5 | 97.2 | 0.52 | 2.01 | 0.98 | 1.08 | 4.59 | 6.70 |
| | | 5 | M | 10.4 | 96.3 | 0.43 | 2.87 | 1.10 | 1.18 | 5.58 | 6.73 |
| | | 6 | M | 10.2 | 94.4 | 0.63 | 3.06 | 1.01 | 1.25 | 5.95 | 6.75 |
| DPT - | Initial | | | 10.7 | 100 | 0.18 | 0.76 | 0.94 | 0.89 | 2.77 | 6.72 |
| 10 mg/ml | 10° C. | 0.5 | M | 10.8 | 101 | 0.65 | 1.23 | 0.72 | 0.82 | 3.42 | 6.80 |
| Magnesium | | 1 | M | 10.7 | 100 | 0.42 | 1.56 | 0.68 | 0.96 | 3.62 | 6.76 |
| hydroxide | | 1.5 | M | 10.6 | 99.1 | 0.60 | 1.95 | 0.68 | 0.90 | 4.13 | 6.73 |
| Calcium | | 2 | M | 10.2 | 95.3 | 0.80 | 2.40 | 0.71 | 1.17 | 5.08 | 6.76 |
| hydroxide | | 2.5 | M | 10.1 | 94.4 | 0.78 | 2.60 | 0.71 | 0.88 | 4.97 | 6.74 |
| pH - 6.75 | | 3 | M | 10.1 | 94.4 | 1.42 | 3.26 | 0.79 | 1.34 | 6.81 | 6.68 |
| | 5° C. | 1 | M | 10.8 | 101 | 0.33 | 1.12 | 0.99 | 0.98 | 3.42 | 6.70 |
| | | 2 | M | 10.6 | 99.1 | 0.45 | 1.60 | 0.60 | 1.19 | 3.84 | 6.75 |
| | | 3 | M | 10.3 | 96.3 | 0.73 | 2.03 | 0.56 | 1.06 | 4.38 | 6.81 |
| | | 5 | M | 10.3 | 96.3 | 0.59 | 2.54 | 0.68 | 0.85 | 4.66 | 6.77 |
| | | 6 | M | 10.3 | 96.3 | 0.94 | 2.80 | 0.64 | 1.00 | 5.38 | 6.75 |

As shown in Table 3, the addition of Mg(OH)₂ stabilized the daptomycin-containing solutions between pH 6.5 and 6.75. The area % of the total impurities increased about 1.61% over three months storage and 2.61% after six months storage at 5° C. at a pH of 6.75. Such an increase projects a shelf-life of about 18 months under refrigerated conditions.

The area % of the total impurities increased about 1.82% over three months storage and 3.18% after six months storage at 5° C. at a pH of 6.5, which also projects a shelf-life of about 18 months.

It can be seen that these formulations are also therefore within the scope of the invention since they are expected to have long term stability and low levels of impurities when stored for the time periods of at least about 18 months at temperatures below 25° C.

### Example 4

Daptomycin (“DPT”) was dissolved in distilled water to obtain a daptomycin concentration of 10 mg/mL and Ca(OH)₂ was added to obtain a Ca(OH)₂ concentration of 0.68 mg/ml. The pH was adjusted with 0.1N NaOH to obtain a pH as indicated in Table 4 below. Samples were made isotonic with the addition of calcium chloride dihydrate or trehalose dihydrate as indicated in Table 4 below. The samples were stored at the temperatures indicated in Table 4 below.

Samples were tested for impurities after initial preparation, and at times indicated in Table 4 below. The samples were tested via HPLC, at a wavelength of 223 nm, and the amount of daptomycin in the initial sample and the relative retention times (“RRT”) for each of the hydrolysis product of daptomycin (0.66), the β-isomer of daptomycin (0.97) and anhydro-daptomycin (1.1) were added to obtain the total impurities area-under-the-curve (“AUC”) after storage. The test data is reported in Table 4 below.

TABLE 4
Stability of Daptomycin (10 mg/mL) in presence of Ca(OH)₂ and NaOH,
with and without Tonicifying Agents

| | | Time | Conc. | % of | % Area of degradants | | | | % of | pH |
|---|---|---|---|---|---|---|---|---|---|---|
| Formulation | Temp. | Period | (mg/mL) | Initial | ΣROP | β | AH DPT | Σunknown | Total | Sample |
| DPT - | Initial | | 10.6 | 100 | 0.06 | 0.57 | 0.72 | 0.64 | 1.99 | 7.03 |
| 10 mg/mL | 15° C. | 1 M | 10.1 | 95.3 | 0.93 | 2.24 | 0.38 | 0.77 | 4.32 | 7.25 |
| Ca(OH)₂ - | | 2 M | 9.94 | 93.8 | 1.90 | 3.22 | 0.58 | 1.39 | 7.09 | 7.23 |
| 0.68 mg/mL | | 3 M | 9.36 | 88.3 | 2.91 | 4.38 | 0.60 | 1.68 | 9.57 | 7.24 |
| Water qs to | 10° C. | 1 M | 10.4 | 98.1 | 0.70 | 1.96 | 0.38 | 0.74 | 3.78 | 7.20 |
| 1 mL | | 2 M | 10.2 | 96.2 | 1.37 | 2.49 | 0.38 | 1.24 | 5.48 | 7.21 |
| pH - 7.00 with | | 3 M | 10.0 | 94.3 | 2.09 | 3.25 | 0.51 | 1.21 | 7.06 | 7.27 |
| 0.1N NaOH | | 4 M | 9.85 | 92.9 | 2.22 | 3.69 | 0.64 | 1.56 | 8.11 | 7.31 |
| | | 5 M | 9.74 | 91.9 | 2.00 | 4.29 | 0.84 | 1.81 | 8.94 | 7.28 |
| | 5° C. | 1 M | 10.6 | 100 | 0.41 | 1.50 | 0.38 | 0.78 | 3.07 | 7.23 |
| | | 2 M | 10.2 | 96.2 | 0.66 | 1.80 | 0.47 | 1.12 | 4.05 | 7.26 |
| | | 3 M | 10.0 | 94.3 | 0.48 | 2.06 | 0.65 | 1.26 | 4.45 | 7.23 |
| | | 4 M | 10.0 | 94.3 | 0.76 | 2.20 | 0.55 | 1.02 | 4.53 | 7.31 |
| | | 5 M | 9.99 | 94.2 | 1.45 | 2.73 | 0.44 | 1.35 | 5.97 | 7.27 |
| DPT - | Initial | | 10.6 | 100 | 0.05 | 0.50 | 0.92 | 0.66 | 2.13 | 7.28 |
| 10 mg/mL | 15° C. | 1 M | 10.2 | 96.2 | 0.73 | 2.21 | 0.60 | 0.76 | 4.30 | 7.20 |
| Ca(OH)₂ - | | 2 M | 9.93 | 93.7 | 1.34 | 3.33 | 0.76 | 1.31 | 6.74 | 7.23 |
| 0.68 mg/mL | | 3 M | 9.61 | 90.7 | 2.22 | 4.50 | 0.78 | 1.66 | 9.16 | 7.10 |
| Water qs to | 10° C. | 1 M | 10.3 | 97.2 | 0.53 | 1.92 | 0.58 | 0.83 | 3.86 | 7.13 |
| 1 mL | | 2 M | 10.1 | 95.3 | 1.01 | 2.62 | 0.57 | 1.25 | 5.45 | 7.10 |
| pH - 6.75 with | | 3 M | 10.0 | 94.3 | 1.49 | 3.31 | 0.65 | 1.22 | 6.67 | 7.14 |
| 0.1N NaOH | | 4 M | 9.75 | 92.0 | 1.64 | 3.90 | 0.70 | 1.38 | 7.62 | 7.17 |
| | | 5 M | 9.69 | 91.4 | 1.41 | 4.55 | 1.01 | 1.77 | 8.74 | 7.14 |
| | 5° C. | 1 M | 10.3 | 97.2 | 0.32 | 1.35 | 0.65 | 0.77 | 3.09 | 7.10 |
| | | 2 M | 10.2 | 96.2 | 0.47 | 1.73 | 0.71 | 1.13 | 4.04 | 7.12 |
| | | 3 M | 10.2 | 96.2 | 0.44 | 2.08 | 0.65 | 1.36 | 4.53 | 7.25 |
| | | 4 M | 10.1 | 95.3 | 0.46 | 2.32 | 0.77 | 1.40 | 4.95 | 7.29 |
| | | 5 M | 9.99 | 94.2 | 0.95 | 3.01 | 0.61 | 1.06 | 5.63 | 7.27 |
| DPT - | Initial | | 10.5 | 100 | 0.05 | 0.55 | 0.71 | 0.59 | 1.90 | 7.00 |
| 10 mg/mL | 15° C. | 1 M | 10.2 | 97.1 | 0.67 | 1.84 | 0.22 | 0.75 | 3.48 | 6.76 |
| Ca(OH)₂ - | | 2 M | 10.0 | 95.2 | 1.30 | 2.36 | 0.38 | 1.22 | 5.26 | 6.75 |
| 0.68 mg/mL | | 3 M | 9.84 | 93.7 | 2.03 | 3.00 | 0.37 | 1.55 | 6.95 | 6.72 |
| CaCl₂•2H₂O - | 10° C. | 1 M | 10.3 | 98.1 | 0.42 | 1.59 | 0.21 | 0.71 | 2.93 | 6.76 |
| 16.2 mg/mL | | 2 M | 10.3 | 98.1 | 0.88 | 1.88 | 0.21 | 1.07 | 4.04 | 6.74 |
| Water qs to | | 3 M | 10.2 | 97.1 | 1.33 | 2.30 | 0.32 | 1.10 | 5.05 | 6.71 |
| 1 mL | | 4 M | 10.1 | 96.2 | 1.39 | 2.48 | 0.33 | 1.16 | 5.36 | 6.75 |
| pH - 6.75 with | | 5 M | 10.1 | 96.2 | 1.30 | 2.75 | 0.31 | 1.34 | 5.70 | 6.78 |
| 0.1N NaOH | 5° C. | 1 M | 10.4 | 99.0 | 0.32 | 1.39 | 0.27 | 0.78 | 2.76 | 6.74 |
| | | 2 M | 10.3 | 98.1 | 0.40 | 1.58 | 0.37 | 1.05 | 3.40 | 6.73 |
| | | 3 M | 10.2 | 97.1 | 0.46 | 1.67 | 0.37 | 1.33 | 3.83 | 6.77 |
| | | 4 M | 10.2 | 97.1 | 0.42 | 1.73 | 0.46 | 1.31 | 3.92 | 6.73 |
| | | 5 M | 10.1 | 96.2 | 0.70 | 2.09 | 0.30 | 0.97 | 4.06 | 6.75 |
| DPT - | Initial | | 10.5 | 100 | 0.07 | 0.49 | 0.84 | 0.59 | 1.99 | 6.75 |
| 10 mg/mL | 15° C. | 1 M | 10.1 | 96.2 | 0.78 | 2.18 | 0.53 | 0.81 | 4.30 | 7.13 |
| Ca(OH)₂ - | | 2 M | 9.90 | 94.3 | 1.58 | 3.25 | 0.67 | 1.30 | 6.80 | 7.15 |
| 0.68 mg/mL | | 3 M | 9.65 | 91.9 | 2.39 | 4.42 | 0.71 | 1.69 | 9.21 | 7.10 |
| Trehalose•2H₂O - | 10° C. | 1 M | 10.4 | 99.0 | 0.55 | 1.66 | 0.51 | 0.65 | 3.37 | 7.08 |
| 50 mg/mL | | 2 M | 10.1 | 96.2 | 0.99 | 2.38 | 0.50 | 1.21 | 5.08 | 7.08 |
| Water qs to | | 3 M | 10.0 | 95.2 | 1.56 | 3.09 | 0.62 | 1.25 | 6.52 | 7.10 |
| 1 mL | | 4 M | 9.87 | 94.0 | 1.62 | 3.59 | 0.69 | 1.31 | 7.21 | 7.15 |
| pH - 6.75 with | | 5 M | 9.71 | 92.5 | 1.56 | 4.14 | 1.04 | 1.95 | 8.69 | 7.19 |
| 0.1N NaOH | 5° C. | 1 M | 10.4 | 99.0 | 0.40 | 1.32 | 0.57 | 0.76 | 3.05 | 7.03 |
| | | 2 M | 10.2 | 97.1 | 0.55 | 1.66 | 0.60 | 1.13 | 3.94 | 7.07 |
| | | 3 M | 10.0 | 95.2 | 0.51 | 1.97 | 0.68 | 1.34 | 4.50 | 7.14 |
| | | 4 M | 10.0 | 95.2 | 0.56 | 2.20 | 0.79 | 1.05 | 4.60 | 7.19 |
| | | 5 M | 10.0 | 95.2 | 0.94 | 2.80 | 0.64 | 1.16 | 5.54 | 7.18 |

As shown in Table 4, the sample with Ca(OH)₂, CaCl₂ and pH 6.75 with NaOH exhibited an increase in the area % of the total impurities of about 1.93% over three months analysis and 2.16% over five months analysis at 5° C. Such an increase projects a shelf-life of about 18 months under refrigerated conditions with levels of impurities within the levels required herein. This sample also exhibited little fluctuation in pH.

The sample with Ca(OH)₂ and pH 7 with NaOH exhibited an increase in the area % of the total impurities of about 2.46% over three months storage and 3.98% over five months storage at 5° C. The sample with Ca(OH)₂ and pH 6.75 with NaOH exhibited an increase in the area % of the total impurities of about 2.4% over three months storage and 3.5% over five months storage at 5° C. The sample with Ca(OH)₂, Trehalose and pH 6.75 with NaOH exhibited an increase in the area % of the total impurities of about 2.51% over three months storage and 3.55% over five months storage at 5° C. Daptomycin-containing compositions with such high levels of degradation would not be suitable for long-term storage.

### Example 5

Daptomycin (“DPT”) was dissolved in distilled water to obtain a daptomycin concentration of 10 mg/mL. Calcium chloride was added to the daptomycin-containing solution in amounts indicated in Table 5 below. The pH was adjusted to 6.75 with NaOH as indicated in Table 5 below. The volume of the solution was adjusted to 1 mL with water. The samples were stored at the temperatures indicated in Table 5 below.

Samples were tested for impurities after initial preparation, and at times indicated in Table 5. The samples were tested via HPLC, at a wavelength of 223 nm, and the amount of daptomycin in the initial sample and the relative retention times (“RRT”) for each of the hydrolysis product of daptomycin (0.66), the β-isomer of daptomycin (0.97) and anhydro-daptomycin (1.1) were added to obtain the total impurities area-under-the-curve (“AUC”) after storage. The test data is reported in Table 5 below.

TABLE 5
Stability of Daptomycin (10 mg/mL) in presence of CaCl₂ and NaOH

| | | Time | Conc. | % of | RRTs of Degradants | | | | % of | pH |
|---|---|---|---|---|---|---|---|---|---|---|
| Formulation | Temp. | Period | (mg/mL) | Initial | ΣROP | β | AH DPT | Σunknown | Total | Sample |
| DPT - | Initial | | 10.2 | 100 | 0.08 | 0.66 | 0.94 | 0.70 | 2.38 | 6.98 |
| 10 mg/mL | 10° C. | 1 M | 9.90 | 97.1 | 0.83 | 2.31 | 1.06 | 1.32 | 5.52 | 6.96 |
| CaCl₂ - | | 2 M | 9.76 | 95.7 | 0.87 | 3.01 | 1.23 | 1.57 | 6.68 | 6.93 |
| 0.5 mg/mL | | 3 M | 9.35 | 91.7 | 2.61 | 5.24 | 1.16 | 1.05 | 10.06 | 6.94 |
| Water qs to | 5° C. | 1 M | 9.95 | 97.5 | 0.49 | 1.60 | 0.73 | 0.76 | 3.58 | 6.99 |
| 1 mL | | 2 M | 9.89 | 97.0 | 0.50 | 1.97 | 0.65 | 0.81 | 3.93 | 6.98 |
| pH - 6.75 | | 3 M | 9.65 | 94.6 | 1.53 | 3.29 | 0.99 | 0.91 | 6.72 | 6.94 |
| with 0.1N | | | | | | | | | | |
| NaOH | | | | | | | | | | |
| DPT - | Initial | | 10.1 | 100 | 0.19 | 0.82 | 0.70 | 0.65 | 2.36 | 6.91 |
| 10 mg/mL | 10° C. | 1 M | 9.91 | 98.1 | 0.80 | 1.99 | 0.69 | 0.93 | 4.41 | 6.87 |
| CaCl₂ - | | 2 M | 9.76 | 96.6 | 0.72 | 2.61 | 1.06 | 1.57 | 5.96 | 6.84 |
| 1 mg/mL | | 3 M | 9.64 | 95.4 | 2.32 | 4.43 | 1.03 | 0.97 | 8.75 | 6.86 |
| Water qs to | 5° C. | 1 M | 10.0 | 99.0 | 0.60 | 1.62 | 0.88 | 1.28 | 4.38 | 6.85 |
| 1 mL | | 2 M | 9.97 | 98.7 | 0.52 | 1.78 | 0.80 | 1.38 | 4.48 | 6.81 |
| pH - 6.75 | | 3 M | 9.73 | 96.3 | 1.51 | 2.89 | 0.86 | 0.90 | 6.16 | 6.83 |
| with 0.1N | | | | | | | | | | |
| NaOH | | | | | | | | | | |
| DPT - | Initial | | 9.86 | 100 | 0.14 | 0.92 | 0.60 | 0.84 | 2.50 | 6.68 |
| 10 mg/mL | 10° C. | 1 M | 9.87 | 100.1 | 0.43 | 1.62 | 0.41 | 0.90 | 3.36 | 6.69 |
| CaCl₂•2H₂O - | | 2 M | 9.84 | 99.8 | 0.54 | 2.07 | 0.39 | 1.14 | 4.14 | 6.71 |
| 4 mg/mL | | 3 M | 9.72 | 98.6 | 0.83 | 2.50 | 0.38 | 0.88 | 4.59 | 6.74 |
| Water-qs to | 5° C. | 1 M | 9.85 | 99.9 | 0.42 | 1.65 | 0.39 | 0.86 | 3.32 | 6.66 |
| 1 mL | | 2 M | 9.79 | 99.3 | 0.47 | 1.92 | 0.49 | 0.86 | 3.74 | 6.69 |
| pH 6.75 | | 3 M | 9.72 | 98.6 | 0.66 | 2.01 | 0.34 | 0.83 | 3.84 | 6.70 |
| with 1N | | | | | | | | | | |
| NaOH | | | | | | | | | | |
| DPT - | Initial | | 10.1 | 100 | 0.10 | 0.84 | 0.71 | 0.92 | 2.57 | 6.59 |
| 10 mg/mL | 10° C. | 1 M | 9.96 | 98.6 | 0.33 | 1.58 | 0.43 | 0.90 | 3.24 | 6.61 |
| CaCl₂•2H₂O - | | 2 M | 9.75 | 96.5 | 0.47 | 1.90 | 0.35 | 1.09 | 3.81 | 6.58 |
| 8 mg/mL | | 3 M | 9.67 | 95.7 | 0.73 | 2.39 | 0.33 | 0.96 | 4.41 | 6.61 |
| Water-qs to | 5° C. | 1 M | 9.96 | 98.6 | 0.34 | 1.60 | 0.36 | 0.90 | 3.20 | 6.60 |
| 1 mL | | 2 M | 9.90 | 98.0 | 0.40 | 1.81 | 0.33 | 0.93 | 3.47 | 6.58 |
| pH 6.75 | | 3 M | 9.87 | 97.7 | 0.56 | 1.87 | 0.25 | 0.83 | 3.51 | 6.59 |
| with 1N | | | | | | | | | | |
| NaOH | | | | | | | | | | |
| DPT - | Initial | | 10.0 | 100 | 0.15 | 0.80 | 0.59 | 0.85 | 2.39 | 6.57 |
| 10 mg/mL | 10° C. | 1 M | 10.0 | 100 | 0.44 | 1.45 | 0.42 | 0.83 | 3.14 | 6.60 |
| CaCl₂•2H₂O - | | 2 M | 9.95 | 99.5 | 0.50 | 1.82 | 0.34 | 0.95 | 3.61 | 6.58 |
| 12 mg/mL | | 3 M | 9.74 | 97.4 | 0.80 | 2.16 | 0.27 | 0.92 | 4.15 | 6.64 |
| Water-qs to | 5° C. | 1 M | 10.1 | 101 | 0.40 | 1.47 | 0.33 | 0.87 | 3.07 | 6.58 |
| 1 mL | | 2 M | 10.1 | 101 | 0.49 | 1.55 | 0.30 | 0.80 | 3.14 | 6.60 |
| pH 6.75 | | 3 M | 10.0 | 100 | 0.67 | 1.76 | 0.23 | 0.79 | 3.45 | 6.62 |
| with 1N | | | | | | | | | | |
| NaOH | | | | | | | | | | |

As shown in Table 5, the daptomycin-containing formulations including greater than 1 mg/ml CaCl₂ and having a pH adjusted to about 6.75 with NaOH exhibited long-term storage stability. The area % of the total impurities increased about 1.34% at a pH of 6.75 over three months analysis at 5° C. in the formulation including 4 mg/ml CaCl₂. The area % of the total impurities increased about 0.94% at a pH of 6.75 over three months analysis at 5° C. in the formulation including 8 mg/ml CaCl₂. The area % of the total impurities increased about 1.06% at a pH of 6.75 over three months analysis at 5° C. in the formulation including 12 mg/ml CaCl₂. Such increases project a shelf-life of at least about 24 months under refrigerated conditions with levels of impurities within the levels required herein. Additionally, these three samples exhibited limited fluctuation of pH.

As shown in Table 5, the area % of the total impurities increased about 4.34% at a pH of 6.75 over three months analysis at 5° C. in the formulation including 0.5 mg/ml CaCl₂. The area % of the total impurities increased about 3.8% at a pH of 6.75 over three months analysis at 5° C. in the formulation including 1 mg/ml CaCl₂. Daptomycin-containing compositions with such high levels of degradation would not be suitable for long-term storage.

### Example 6

Daptomycin (“DPT”) was dissolved in distilled water to obtain a daptomycin concentration of 10 mg/mL. Calcium chloride was added to the daptomycin-containing solution to obtain a concentration of 1.5 mg/ml calcium chloride. The pH was adjusted with 1 N NaOH as indicated in Table 6. The volume of the solution was adjusted to 1 mL with water. The samples were stored at the temperatures indicated in Table 6 below.

Samples were tested for impurities after initial preparation, and as indicated in Table 6. The samples were tested via HPLC, at a wavelength of 223 nm, and the amount of daptomycin in the initial sample and the relative retention times (“RRT”) for each of the hydrolysis product of daptomycin (0.66), the β-isomer of daptomycin (0.97) and anhydro-daptomycin (1.1) were added to obtain the total impurities area-under-the-curve (“AUC”) after storage. The test data is reported in Table 6 below.

TABLE 6
Stability of Daptomycin (10 mg/mL) in presence of CaCl₂ and NaOH

| | | Time | Conc. | % of | RRTs of Degradants | | | | % of | pH |
|---|---|---|---|---|---|---|---|---|---|---|
| Formulation | Temp. | Period | (mg/mL) | Initial | ΣROP | β | AH DPT | Σunknown | Total | Sample |
| DPT - | Initial | | 10.2 | 100 | 0.12 | 0.61 | 1.13 | 0.73 | 2.59 | 6.20 |
| 10 mg/mL | 10° C. | 0.5 M | 10.2 | 100 | 0.18 | 0.98 | 1.30 | 0.80 | 3.26 | 6.18 |
| CaCl₂ - | | 1 M | 9.97 | 97.7 | 0.49 | 1.79 | 1.47 | 0.99 | 4.74 | 6.22 |
| 1.5 mg/mL | 5° C. | 1 M | 10.1 | 99.0 | 0.38 | 1.24 | 1.13 | 0.95 | 3.70 | 6.21 |
| Water qs to | | | | | | | | | | |
| 1 mL | | | | | | | | | | |
| pH - 6.00 | | | | | | | | | | |
| with 1N | | | | | | | | | | |
| NaOH | | | | | | | | | | |
| DPT - | Initial | | 10.0 | 100 | 0.15 | 0.65 | 1.09 | 0.63 | 2.52 | 6.40 |
| 10 mg/mL | 10° C. | 0.5 M | 9.91 | 99.1 | 0.21 | 1.10 | 1.02 | 0.84 | 3.17 | 6.37 |
| CaCl₂ - | | 1 M | 9.94 | 99.4 | 0.37 | 1.38 | 0.96 | 1.02 | 3.73 | 6.39 |
| 1.5 mg/mL | 5° C. | 1 M | 9.95 | 99.5 | 0.37 | 1.30 | 0.98 | 0.90 | 3.55 | 6.38 |
| Water qs to | | | | | | | | | | |
| 1 mL | | | | | | | | | | |
| pH - 6.25 | | | | | | | | | | |
| with 1N | | | | | | | | | | |
| NaOH | | | | | | | | | | |
| DPT - | Initial | | 10.2 | 100 | 0.17 | 0.65 | 0.99 | 0.57 | 2.38 | 6.64 |
| 10 mg/mL | 10° C. | 0.5 M | 10.2 | 100 | 0.25 | 1.19 | 0.79 | 0.75 | 2.98 | 6.61 |
| CaCl₂ - | | 1 M | 10.2 | 100 | 0.51 | 1.34 | 0.80 | 0.77 | 3.42 | 6.60 |
| 1.5 mg/mL | 5° C. | 1 M | 10.1 | 99.0 | 0.43 | 1.34 | 0.61 | 0.91 | 3.29 | 6.66 |
| Water qs to | | | | | | | | | | |
| 1 mL | | | | | | | | | | |
| pH - 6.50 | | | | | | | | | | |
| with 1N | | | | | | | | | | |
| NaOH | | | | | | | | | | |
| DPT - | Initial | | 10.3 | 100 | 0.23 | 0.84 | 0.73 | 0.78 | 2.58 | 6.96 |
| 10 mg/mL | 10° C. | 0.5 M | 10.3 | 100 | 0.38 | 1.38 | 0.42 | 0.74 | 2.92 | 6.92 |
| CaCl₂ - | | 1 M | 10.1 | 98.1 | 0.61 | 1.59 | 0.41 | 0.86 | 3.47 | 6.89 |
| 1.5 mg/mL | 5° C. | 1 M | 10.2 | 99.0 | 0.60 | 1.50 | 0.41 | 0.85 | 3.36 | 6.95 |
| Water qs to | | | | | | | | | | |
| 1 mL | | | | | | | | | | |
| pH - 7.00 | | | | | | | | | | |
| with 1N | | | | | | | | | | |
| NaOH | | | | | | | | | | |

As shown in Table 6, the daptomycin-containing formulations including greater than 1 mg/ml CaCl₂ and having a pH adjusted to about 6.0 to 7.0 with NaOH exhibited long-term storage stability.

The area % of the total impurities increased about 1.11% over one month analysis at 5° C. at a pH of 6.0. The area % of the total impurities increased about 1.03% over one month analysis at 5° C. at a pH of 6.25. The area % of the total impurities increased about 0.91% over one month analysis at 5° C. at a pH of 6.5. The area % of the total impurities increased about 0.78% over one month analysis at 5° C. at a pH of 7. These increases are comparable to the increases observed in Example 6 above. Such increases project a shelf-life of at least about 18 months under refrigerated conditions with levels of impurities within the levels required herein. Additionally, these samples exhibited limited fluctuation of pH.

### Example 7

Daptomycin (“DPT”) was dissolved in 0.1M calcium lactate to obtain a daptomycin concentration of 10 mg/mL. The pH was adjusted as indicated in Table 7 with 1N NaOH. The samples were stored at the temperatures indicated in Table 7 below.

Samples were tested for impurities after initial preparation, and as indicated in Table 7. The samples were tested via HPLC, at a wavelength of 223 nm, and the amount of daptomycin in the initial sample and the relative retention times (“RRT”) for each of the hydrolysis product of daptomycin (0.66), the β-isomer of daptomycin (0.97) and anhydro-daptomycin (1.1) were added to obtain the total impurities area-under-the-curve (“AUC”) after storage. The test data is reported in Table 7 below.

TABLE 7
Stability of Daptomycin (10 mg/mL) with 0.1M Calcium Lactate and 1N NaOH

| | | Time | Conc. | % of | RRTs of Degradants | | | | % of | pH of |
|---|---|---|---|---|---|---|---|---|---|---|
| Formulation | Temp. | Period | (mg/mL) | Initial | ΣROP | β | AH DPT | Σunknown | Total | Sample |
| DPT - | Initial | | 10.3 | 100 | 0.08 | 0.56 | 1.06 | 0.80 | 2.50 | 6.2 |
| 10 mg/mL | 10° C. | 1 M | 10.0 | 97.1 | 0.44 | 1.61 | 1.05 | 1.12 | 4.22 | 6.23 |
| Calcium | | 2 M | 9.86 | 95.7 | 0.36 | 2.18 | 1.24 | 2.10 | 5.88 | 6.25 |
| Lactate - | | 3 M | 9.69 | 94.1 | 0.78 | 3.16 | 1.05 | 0.87 | 5.86 | 6.29 |
| 0.1M | | 4 M | 9.54 | 92.6 | 0.64 | 3.72 | 1.20 | 1.49 | 7.05 | 6.18 |
| Water qs to | 5° C. | 1 M | 10.3 | 100 | 0.28 | 1.19 | 1.00 | 1.00 | 3.47 | 6.20 |
| 1 mL | | 2 M | 10.2 | 99.0 | 0.30 | 1.81 | 1.21 | 1.62 | 4.94 | 6.21 |
| pH - 6.25 | | 3 M | 9.97 | 96.8 | 0.51 | 2.30 | 1.33 | 1.29 | 5.43 | 6.26 |
| with NaOH | | 4 M | 10.0 | 97.1 | 0.42 | 2.49 | 1.35 | 1.65 | 5.91 | 6.23 |
| DPT - | Initial | | 10.0 | 100 | 0.07 | 0.56 | 1.03 | 0.85 | 2.51 | 6.47 |
| 10 mg/mL | 10° C. | 1 M | 9.91 | 97.2 | 0.48 | 1.72 | 0.72 | 1.04 | 3.96 | 6.42 |
| Calcium | | 2 M | 9.87 | 96.8 | 0.38 | 2.12 | 0.96 | 1.28 | 4.74 | 6.43 |
| Lactate - | | 3 M | 9.69 | 95.0 | 0.90 | 2.97 | 0.64 | 0.79 | 5.3 | 6.46 |
| 0.1M | | 4 M | 9.67 | 94.8 | 0.70 | 3.25 | 0.92 | 1.72 | 6.59 | 6.40 |
| Water qs to | 5° C. | 1 M | 10.0 | 98.0 | 0.34 | 1.29 | 0.79 | 0.98 | 3.40 | 6.52 |
| 1 mL | | 2 M | 9.88 | 96.9 | 0.32 | 1.75 | 0.63 | 0.94 | 3.64 | 6.53 |
| pH - 6.50 | | 3 M | 10.0 | 98.0 | 0.61 | 2.15 | 0.66 | 1.01 | 4.33 | 6.57 |
| with NaOH | | 4 M | 9.79 | 96.0 | 0.48 | 2.35 | 0.67 | 1.23 | 4.73 | 6.59 |

As shown in Table 7, the daptomycin-containing formulations including 0.1M calcium lactate and having a pH adjusted to about 6.5 with NaOH exhibited long-term storage stability. The area % of the total impurities increased about 1.81% over three months analysis and 2.22% over four months analysis at 5° C. in the formulation including 0.1M calcium lactate and having a pH adjusted to about 6.5 with NaOH. Such an increase projects a shelf-life of about 18 months under refrigerated conditions with levels of impurities within the levels required herein.

The area % of the total impurities increased about 2.93% over three months analysis and 3.41% over four months storage at 5° C. in the formulation including 0.1M calcium lactate and having a pH adjusted to about 6.25 with NaOH. Daptomycin-containing compositions with such high levels of degradation would not be suitable for long-term storage.

We claim:

1. A long term storage stable daptomycin-containing composition, comprising a pharmacologically acceptable fluid comprising:
a) daptomycin or a pharmaceutically acceptable salt thereof at a concentration of less than or equal to about 25 mg/mL; and
b) a source of calcium selected from the group consisting of calcium chloride and calcium lactate;
said daptomycin-containing composition having a pH of from about 6 to about 7, and
wherein total impurities are less than about 10% area-under-the-curve (“AUC”) as determined by high performance liquid chromatography (“HPLC”) at a wavelength of 223 nm, after at least about 18 months of storage at a temperature of from about 5° C. to about 25° C.

2. The long term storage stable daptomycin-containing composition of claim 1, wherein the less than about 10% total impurities include less than about 5% of the hydrolysis product of daptomycin, less than about 5% of the β-isomer of daptomycin and less than about 5% of anhydro-daptomycin based on the amount of daptomycin included in said composition.

3. The long term storage stable daptomycin-containing composition of claim 1, wherein the source of calcium is calcium chloride, and the calcium chloride concentration is greater than 1 mg/ml.

4. The long term storage stable daptomycin-containing composition of claim 3, wherein the calcium chloride concentration is from about 1.5 mg/mL to about 17 mg/mL.

5. The long term storage stable daptomycin-containing composition of claim 4, wherein the calcium chloride concentration is from about 4 mg/mL to about 16.2 mg/mL.

6. The long term storage stable daptomycin-containing composition of claim 5, wherein the calcium chloride concentration is from about 8 mg/mL to about 12 mg/mL.

7. The long term storage stable daptomycin-containing composition of claim 6, wherein the calcium chloride concentration is about 8 mg/mL.

8. The long term storage stable daptomycin-containing composition of claim 6, wherein the calcium chloride concentration is about 12 mg/mL.

9. The long term storage stable daptomycin-containing composition of claim 5, wherein the calcium chloride concentration is about 16.2 mg/mL.

10. The long term storage stable daptomycin-containing composition of claim 1, wherein the source of calcium is calcium lactate, and the calcium lactate concentration is about 0.05M to about 0.3 M.

11. The long term storage stable daptomycin-containing composition of claim 10, wherein the source of calcium is calcium lactate, and the calcium lactate concentration is about 0.1M to about 0.25M.

12. The long term storage stable daptomycin-containing composition of claim 11, wherein the source of calcium is calcium lactate, and the calcium lactate concentration is about 0.1M.

13. The long term storage stable daptomycin-containing composition of claim 1, further comprising a pH adjusting agent.

14. The long term storage stable daptomycin-containing composition of claim 13, wherein the pH adjusting agent is selected from the group consisting of sodium hydroxide and calcium hydroxide.

15. The long term storage stable daptomycin-containing composition of claim 1, wherein the pH of the composition is from about 6.25 to about 6.75.

16. The long term storage stable daptomycin-containing composition of claim 15, wherein the pH of the composition is from about 6.5 to about 6.75.

17. The long term storage stable daptomycin-containing composition of claim 16, wherein the pH of the composition is about 6.5.

18. The long term storage stable daptomycin-containing composition of claim 16, wherein the pH of the composition is about 6.75.

19. The long term storage stable daptomycin-containing composition of claim 1, wherein the composition is maintained at a temperature of from about 5° C. to about 15° C.

20. The long term storage stable daptomycin-containing composition of claim 19, wherein the composition is maintained at a temperature of from about 5° C. to about 10° C.

21. The long term storage stable daptomycin-containing composition of claim 20, wherein the composition is maintained at a temperature of about 5° C.

22. The long term storage stable daptomycin-containing composition of claim 1, wherein the daptomycin concentration is from about 1 mg/mL to about 25 mg/mL.

23. The long term storage stable daptomycin-containing composition of claim 22, wherein the daptomycin concentration is from about 5 mg/mL to about 20 mg/mL.

24. The long term storage stable daptomycin-containing composition of claim 23, wherein the daptomycin concentration is from about 7.5 mg/mL to about 15 mg/mL.

25. The long term storage stable daptomycin-containing composition of claim 24, wherein the daptomycin concentration is about 10 mg/mL.

26. The long term storage stable daptomycin-containing composition of claim 1, wherein said long term storage is at least about 2 years.

27. The long term storage stable daptomycin-containing composition of claim 1, further comprising an alkaline hydroxide selected from the group consisting of calcium hydroxide, magnesium hydroxide and aluminum hydroxide.

28. The long term storage stable daptomycin-containing composition of claim 27, wherein the alkaline hydroxide is calcium hydroxide at a concentration of about 0.5 mg/ml to about 1 mg/ml.

29. The long term storage stable daptomycin-containing composition of claim 28, wherein the calcium hydroxide concentration is about 0.68 mg/ml.

30. A long term storage stable daptomycin-containing composition, comprising a pharmacologically acceptable fluid comprising:
a) daptomycin or a pharmaceutically acceptable salt thereof at a concentration of less than or equal to about 25 mg/mL; and
b) calcium chloride at a concentration of greater than 1 mg/mL;
said daptomycin-containing composition having a pH of from about 6 to about 7, and
wherein total impurities are less than about 10% area-under-the-curve (“AUC”) as determined by high performance liquid chromatography (“HPLC”) at a wavelength of 223 nm, after at least about 18 months storage at a temperature of from about 5° C. to about 25° C.

31. The long term storage stable daptomycin-containing composition of claim 30, wherein less than about 10% total impurities include less than about 5% of the hydrolysis product of daptomycin, less than about 5% of the β-isomer of daptomycin and less than about 5% of anhydro-daptomycin based on the amount of daptomycin included in said composition.

32. A long term storage stable daptomycin-containing composition, comprising a pharmacologically acceptable fluid comprising:
a) daptomycin or a pharmaceutically acceptable salt thereof at a concentration of less than or equal to about 25 mg/mL; and
b) 0.1 M calcium lactate;
said daptomycin-containing composition having a pH of about 6.5, and
wherein total impurities are less than about 10% area-under-the-curve (“AUC”) as determined by high performance liquid chromatography (“HPLC”) at a wavelength of 223 nm, after at least about 18 months storage at a temperature of from about 5° C. to about 25° C.

33. The long term storage stable daptomycin-containing composition of claim 32, wherein less than about 10% total impurities include less than about 5% of the hydrolysis product of daptomycin, less than about 5% of the β-isomer of daptomycin and less than about 5% of anhydro-daptomycin based on the amount of daptomycin included in said composition.

34. A long term storage stable daptomycin-containing composition, comprising a pharmacologically acceptable fluid comprising:
a) daptomycin or a pharmaceutically acceptable salt thereof at a concentration of less than or equal to about 25 mg/mL;
b) calcium chloride at a concentration of about 16.2 mg/mL; and
c) calcium hydroxide at a concentration of about 0.68 mg/mL;
said daptomycin-containing composition having a pH of from about 6 to about 7, and
wherein total impurities are less than about 10% area-under-the-curve (“AUC”) as determined by high performance liquid chromatography (“HPLC”) at a wavelength of 223 nm, after at least about 18 months storage at a temperature of from about 5° C. to about 25° C.

35. The long term storage stable daptomycin-containing composition of claim 34, wherein less than about 10% total impurities include less than about 5% of the hydrolysis product of daptomycin, less than about 5% of the β-isomer of daptomycin and less than about 5% of anhydro-daptomycin based on the amount of daptomycin included in said composition.

36. A method of treating a daptomycin sensitive disease in mammals, comprising administering an effective amount of a long term storage stable daptomycin-containing composition of claim 1 to a mammal in need thereof.
